(19) United States
(12) Patent Application Publication
(10) Pub. No.: US 2012/0305000 A1
(43) Pub. Date: Dec. 6, 2012

(54) AdaptAir: A Universal Fit Nasal Interface

(75) Inventors: David Philip Janka, San Francisco, CA (US); Alejandro Palandjoglou, Stanford, CA (US); Neil Mehta, Great Falls, VA (US); Elizabeth Zambricki, Palo Alto, CA (US)

(73) Assignee: The Board of Trustees of the Leland Stanford Junior University, Palo Alto, CA (US)

(21) Appl. No.: 13/487,187

(22) Filed: Jun. 2, 2012

Related U.S. Application Data
(60) Provisional application No. 61/492,378, filed on Jun. 2, 2011.

(51) Int. Cl. A61M 15/08; A61M 16/06

(52) U.S. Cl. 128/205.25

(57) ABSTRACT

A nasal interface and method for delivering continuous positive airway pressure in newborns and infants are provided herein using a universal fit nasal interface that is easily adapted and operated together with a generic nasal cannula.

### 1. RELATED APPLICATION

This application claims priority and other benefits from U.S. Provisional Patent Application Ser. No. 61/492,378, filed Jun. 2, 2011, entitled “Universal fit nasal interface”. Its entire content is specifically incorporated herein by reference.

### 2. BACKGROUND

Surprising to many outside of the global public health sphere, pneumonia is the leading killer of children under 5 years old. At over 2 million deaths per year, pneumonia is responsible for greater annual childhood mortality than diarrheal diseases (1.7 million) and, furthermore, exceeds the combined mortality from malaria (800,000), measles (400,000), and AIDS (300,000). Pneumonia has the potential to be a fatal illness because, in severe forms, accumulation of fluid in the lungs compromises a child's ability to breathe. The delivery of continuously pressurized oxygen is crucial for survival of children with severe pneumonia in order to keep their lungs open.

A therapy known as Bubble CPAP (continuous positive airway pressure) is a simple and affordable way to deliver effective respiratory treatment for children. A fundamental feature of effective Bubble CPAP is that the nasal interface that delivers pressurized oxygen to the baby fits well and does not allow oxygen to escape. Failure to appropriately fit nasal prongs can also lead to complications such as nasal mucosa damage caused by excess movement of the prongs.

Unfortunately, there are currently limited options in the developing world for an effective nasal interface as the customized options available in industrialized nations are prohibitively expensive. Physicians, nurses and respiratory therapists in the developing world say that a poor seal is often their greatest challenge when trying to administer Bubble CPAP. Without an effective nasal seal, children with severe pneumonia are not getting the pressurized gas delivery they need, leading to longer hospitalizations, wasted resources and increased mortality from pneumonia.

AdaptAir is the missing link in effective respiratory care for dying children in the developing world. Based on the simple concept of a one-size-fits-all adapter, AdaptAir is a tremendous improvement over existing, conventional devices that serve for the delivery of nasal continuous positive airway pressure in newborns and infants, since it does not require a complementary system to achieve airtightness and operability. Instead, AdaptAir is designed on one end to fit any generic nasal cannula routinely used worldwide to deliver pressurized oxygen in resource-constrained hospitals. On the other end, conically shaped elements are designed to fit a variety of children's nostrils and create a strong seal. Resource constrained hospitals can continue to use the current materials employed to administer Bubble CPAP therapy with this simple and inexpensive add-on to provide an effective seal. AdaptAir is furthermore made from soft, gentle, pliable silicone allowing it to fit comfortably and safely. Resource-limited settings also reuse the majority of medical devices and products so AdaptAir has been designed for easy cleaning and sterilization.

### 3. SUMMARY

A nasal interface and method for delivering continuous positive airway pressure in newborns and infants are provided herein using a universal fit nasal interface that is easily adapted and operated together with generic nasal cannula. The device and method are an important improvement over existing, conventional devices, since they do not require a complementary system to achieve airtightness and operability. Instead, AdaptAir is designed to fit and successfully operate with any generic nasal cannula.

### 4. BRIEF DESCRIPTION OF THE DRAWINGS

The accompanying drawings illustrate embodiments of the invention and, together with the description, serve to explain the invention. These drawings are offered by way of illustration and not by way of limitation; it is emphasized that the various features of the drawings may not be to-scale.

FIG. 1A illustrates a schematic representation of the anterior view of the universal fit nasal interface. 101 refers to the anterior openings where a nasal cannula is inserted. 103 indicates openings for attachment straps, tape or other material to fasten the nasal interface to the nose of a subject. In one embodiment of the invention, Velcro® straps are used to fasten the nasal interface to the nose of a subject by guiding the straps through the openings 103 and around the subject's head.

FIG. 1B illustrates a schematic representation of the posterior view of the universal fit nasal interface. 102 refers to the conically shaped elements of the nasal interface that are inserted into both narils of a subject, until a snug fit is achieved. The length to which the conically shaped elements are inserted into the narils of a subject depends from the size of the subject's narils. If the subject is a newborn and/or has small-sized narils, the conically shaped elements are inserted deep into the narils, until a snug fit is achieved. In comparison, if the subject has medium or large-sized narils, the conically shaped elements are inserted less deep into the narils, just as much as is needed to obtain a snug fit.

FIG. 1C illustrates a schematic representation of the side view of the universal fit nasal interface.

FIG. 1D illustrates a schematic representation of the posterior view of the universal fit nasal interface.

FIG. 2A illustrates a schematic representation of the top view of the universal fit nasal interface.

FIG. 2B illustrates a schematic representation of the anterior view of the universal fit nasal interface.

FIG. 2C illustrates a schematic representation of the side view of the universal fit nasal interface.

FIG. 3A illustrates a schematic representation of the anterior view of the universal fit nasal interface before the insertion of a standard nasal cannula 104 into the anterior openings 101.

FIG. 3B illustrates a schematic representation of the anterior view of the universal fit nasal interface after the insertion of a standard nasal cannula 104.

FIG. 3C illustrates a schematic representation of the posterior view of the universal fit nasal interface before the insertion of a standard nasal cannula 104.

FIG. 3D illustrates a schematic representation of the side view of the universal fit nasal interface after the insertion of a standard nasal cannula 104.

### 5. DETAILED DESCRIPTION

### 5.1 Universal Fit Nasal Interface and Method For Delivering Continuous Positive Airway Pressure in Newborns and Infants

In resource-limited healthcare environments a system known as Bubble CPAP (continuous positive airway pressure) can be constructed with basic materials for subjects in respiratory distress who require continuously pressurized supplemental oxygen. These materials include an oxygen source, a standard nasal cannula and a container with water. The Bubble CPAP system must have a tight seal at the nasal interface of the subject in order to provide an effective oxygen delivery. A tight seal is confirmed by the presence of consistent bubbling in the water container.

Standard nasal cannulas are generally used in the healthcare field to provide supplemental oxygen to subjects of all ages and health conditions. They are generally and readily available in poor countries. However, they are not designed to fill the nares entirely and thus they allow leakage of the delivered gases and oxygen to occur. Nasal cannulas by themselves are insufficient for providing pressurized supplemental oxygen as it is needed to provide Bubble CPAP, because they allow air leakage to occur. For subjects, who require continuously pressurized supplemental oxygen, nasal cannulas alone are, thus, inefficient. The combination of the universal fit nasal interface with a standard nasal cannula, however, creates an airtight, low-cost and easy to assemble system for providing pressurized supplemental oxygen in the context of Bubble CPAP.

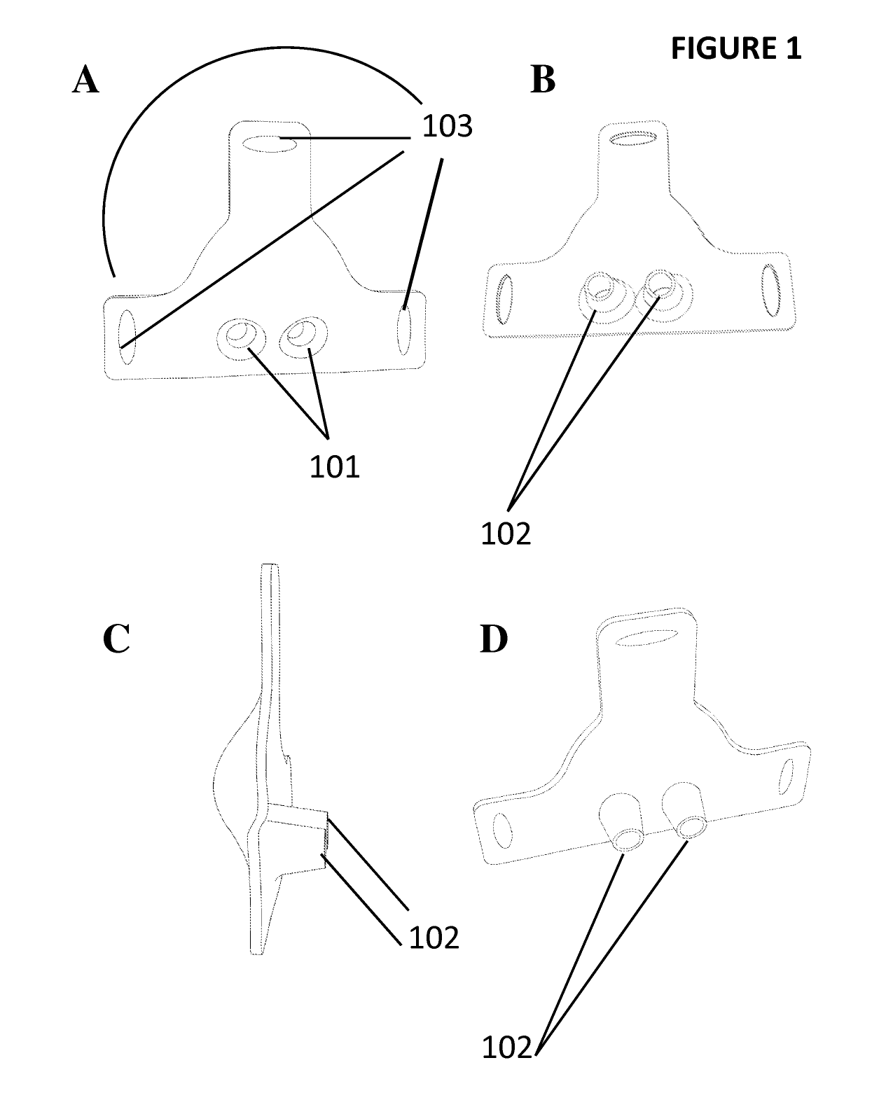

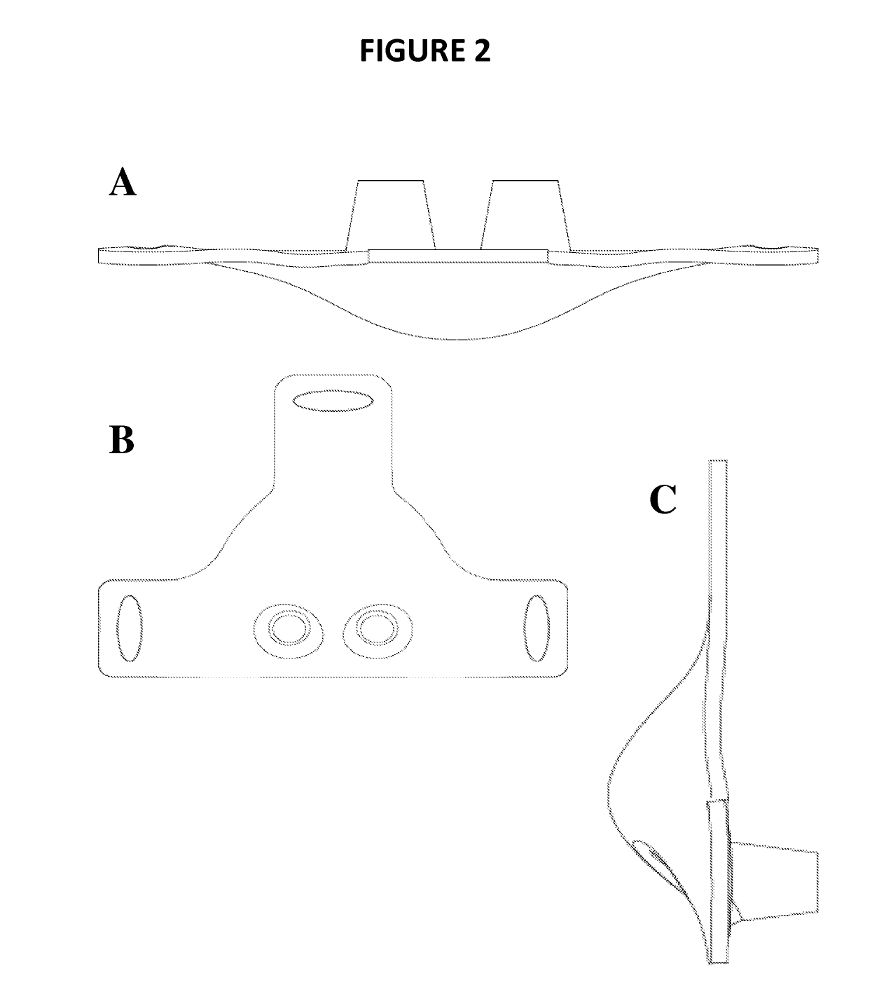

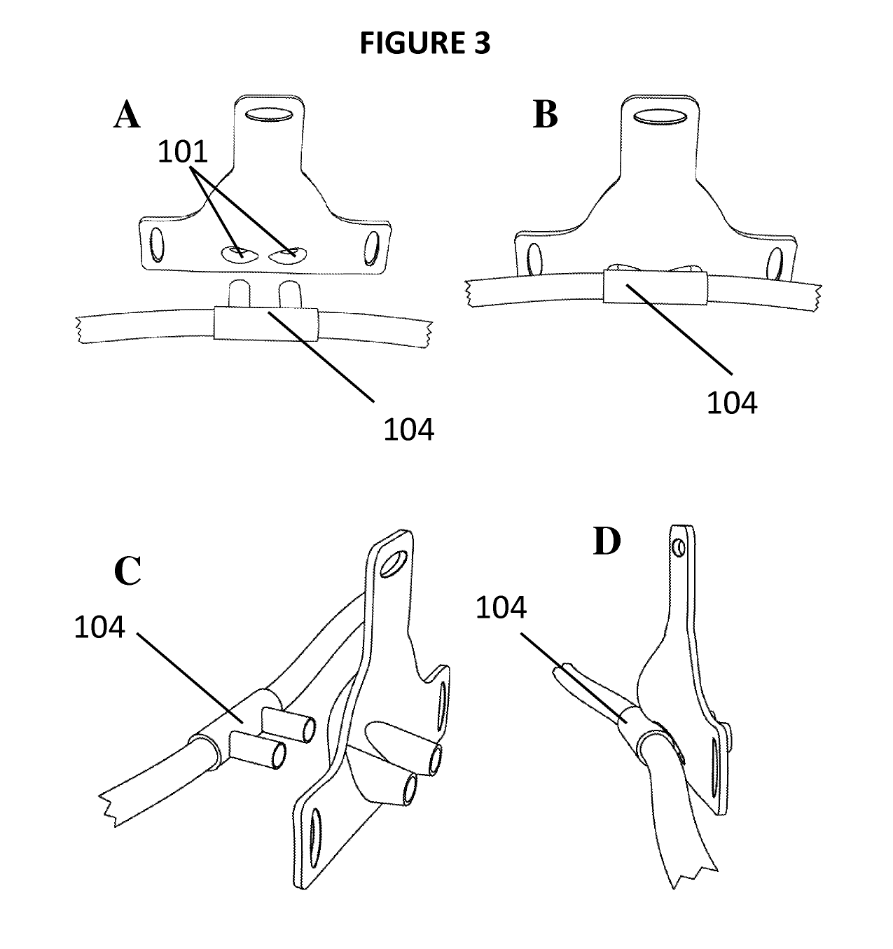

In embodiments of the present invention, the universal fit nasal interface is used with any standard nasal cannula, adjusted in size to fit the prongs of the nasal interface, in order to provide a tight seal at the nares of a subject to deliver continuous pressurized oxygen. A nasal interface, that is size-appropriate for the size and age of the subject, is placed snuggly into the subject's nares and the device is fastened to the subject's head via elastic straps, tape, or other suitable materials, as seen in FIG. 1A. A tight seal is confirmed by consistent bubbling in the water container of the Bubble CPAP system. Such nasal interfaces are particularly appropriate for use in newborns and infants and are sized in accordance to nares of an average-sized newborn and infant, respectively. The improvement of the described universal fit nasal interface and method for delivering continuous pressurized oxygen in newborns and infants lies in the fact that the universal fit nasal interface is easily adaptable to differently sized nares and can be operated together with a standard nasal cannula. The nasal interface and method are an important improvement over existing, conventional devices which require a customized and complementary system to achieve airtightness and operability.

### 5.2 Description of an Embodiment of the Present Invention

The universal fit nasal interface is made out of a flexible material such as silicone or polyethylene which flexes to mold to the contours of most faces of children aged 0-4 years. Any flexible material that meets these qualities is an example of such a material. The flexible material is shaped to cover the nose, creating a nasal interface, with holes around the perimeter of the nasal interface used to insert an elastic band for fastening and keeping the nasal interface in place. Additionally, the nasal interface contains two openings on the anterior surface that pass through to the posterior surface. On the anterior side, these openings accept a standard nasal cannula allowing for the introduction of pressurized gas, such as oxygen or air, into the nasal interface and subsequently, the nose. The openings on the posterior surface of the nasal interface carry conically shaped elements that are to be inserted in a subject's nares, with the conical shape accommodating various-sized nares. The posterior openings directly fit into the nares, thus creating a snug fit in a variety of nares sizes.

What is claimed is:

1. A flexible, universal fit nasal interface for delivering continuous pressurized gas in a subject in need thereof, comprising two extruding, conically shaped openings on one side and two inwards pointed openings on another side, whereby the extruding openings are intended to be inserted into said subject's nares, while the inwards pointed openings are intended to be connected to a standard nasal cannula.

2. The nasal interface according to claim 1, wherein the subject is a newborn or infant.

3. A method for delivering continuous pressurized gas in a subject in need thereof, comprising administering to said subject a flexible, universal fit nasal interface, comprising two extruding, conically shaped openings on one side and two inwards pointed openings on another side, whereby the extruding openings are intended to be inserted into said subject's nares, while the inwards pointed openings are intended to be connected to a standard nasal cannula

4. The method according to claim 3, wherein the subject is a newborn or infant.
